SEQUENCE LISTING (21 sequences)

SEQ ID NO: 1 - protein, 40 aa, Homo sapiens
DAEFRHDSGYEVHHQKLVFFAEDVGSNKGAIIGLMVGGVV

SEQ ID NO: 2 - protein, 42 aa, Homo sapiens [sequence omitted]

SEQ ID NO: 3 - protein, 695 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 4 - protein, 751 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAIPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 5 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 6 - DNA, 2088 bp, Homo sapiens
  exon (1)..(2088)
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 7 - protein, 10 aa, Homo sapiens
AAVTPEERHC

SEQ ID NO: 8 - DNA, 747 bp, Jellyfish
  exon (1)..(747)
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatccaccggatctagataa

SEQ ID NO: 9 - DNA, 29 bp, Artificial Sequence
  Primer
tacggatccgcagaaatacgagaaactgg

SEQ ID NO: 10 - DNA, 30 bp, Artificial Sequence
  Primer
ctgaagctttagggcggacagaagtcggag

SEQ ID NO: 11 - DNA, 28 bp, Artificial Sequence
  Primer
tacggatccccaggcgtccaccagtgag

SEQ ID NO: 12 - DNA, 29 bp, Artificial Sequence
  Primer
tacaagcttcatgacgcaggctacagtgc

SEQ ID NO: 13 - DNA, 960 bp, Homo sapiens
atgtcgtactaccatcaccatcaccatcacgattacgatatcccaacgaccgaaaacctgtattttcagggcgccatggatccgcagaaatacgagaaactggaaaagattggggaaggcacctacggaactgtgttcaaggccaaaaaccgggagactcatgagatcgtggctctgaaacgggtgaggctggatgacgatgatgagggtgtgccgagttccgccctccgggagatctgcctactcaaggagctgaagcacaagaacatcgtcaggcttcatgacgtcctgcacagcgacaagaagctgactttggtttttgaattctgtgaccaggacctgaagaagtattttgacagttgcaatggtgacctcgatcctgagattgtaaagtcattcctcttccagctactaaaagggctgggattctgtcatagccgcaatgtgctacacagggacctgaagccccagaacctgctaataaacaggaatggggagctgaaattggctgattttggcctggctcgagcctttgggattcccgtccgctgttactcagctgaggtggtcacactgtggtaccgcccaccggatgtcctctttggggccaagctgtactccacgtccatcgacatgtggtcagccggctgcatctttgcagagctggccaatgctgggcggcctctttttcccggcaatgatgtcgatgaccagttgaagaggatcttccgactgctggggacgcccaccgaggagcagtggccctctatgaccaagctgccagactataagccctatccgatgtacccggccacaacatccctggtgaacgtcgtgcccaaactcaatgccacagggagggatctgctgcagaaccttctgaagtgtaaccctgtccagcgtatctcagcagaagaggccctgcagcacccctacttctccgacttctgtccgccctaa

SEQ ID NO: 14 - DNA, 576 bp, Homo sapiens
atgtcgtactaccatcaccatcaccatcacgattacgatatcccaacgaccgaaaacctgtattttcagggcgccatggatccccaggcgtccaccagtgagctgcttcgctgcctgggtgagtttctctgccgccggtgctaccgcctgaagcacctgtcccccacggaccccgtgctctggctgcgcagcgtggaccgctcgctgcttctgcagggctggcaggaccagggcttcatcacgccggccaacgtggtcttcctctacatgctctgcagggatgttatctcctccgaggtgggctcggatcacgagctccaggccgtcctgctgacatgcctgtacctctcctactcctacatgggcaacgagatctcctacccgctcaagcccttcctggtggagagctgcaaggaggccttttgggaccgttgcctctctgtcatcaacctcatgagctcaaagatgctgcagataaatgccgacccacactacttcacacaggtcttctccgacctgaagaacgagagcggccaggaggacaagaagcggctcctcctaggcctggatcggtga

SEQ ID NO: 15 - DNA, 879 bp, Homo sapiens
  exon (1)..(879)
atgcagaaatacgagaaactggaaaagattggggaaggcacctacggaactgtgttcaaggccaaaaaccgggagactcatgagatcgtggctctgaaacgggtgaggctggatgacgatgatgagggtgtgccgagttccgccctccgggagatctgcctactcaaggagctgaagcacaagaacatcgtcaggcttcatgacgtcctgcacagcgacaagaagctgactttggtttttgaattctgtgaccaggacctgaagaagtattttgacagttgcaatggtgacctcgatcctgagattgtaaagtcattcctcttccagctactaaaagggctgggattctgtcatagccgcaatgtgctacacagggacctgaagccccagaacctgctaataaacaggaatggggagctgaaattggctaattttggcctggctcgagcctttgggattcccgtccgctgttactcagctgaggtggtcacactgtggtaccgcccaccggatgtcctctttggggccaagctgtactccacgtccatcgacatgtggtcagccggctgcatctttgcagagctggccaatgctgggcggcctctttttcccggcaatgatgtcgatgaccagttgaagaggatcttccgactgctggggacgcccaccgaggagcagtggccctctatgaccaagctgccagactataagccctatccgatgtacccggccacaacatccctggtgaacgtcgtgcccaaactcaatgccacagggagggatctgctgcagaaccttctgaagtgtaaccctgtccagcgtatctcagcagaagaggccctgcagcacccctacttctccgacttctgtccgccctag

SEQ ID NO: 16 - DNA, 30 bp, Artificial Sequence
  Primer
ctgaagcttcgcagaaatacgagaaactgg

SEQ ID NO: 17 - DNA, 30 bp, Artificial Sequence
  Primer
gatctcgagtagggcggacagaagtcggag

SEQ ID NO: 18 - DNA, 33 bp, Artificial Sequence
  Primer
ggagctgaaattggctaattttggcctggctcg

SEQ ID NO: 19 - DNA, 33 bp, Artificial Sequence
  Primer
cgagccaggccaaaattagccaatttcagctcc

SEQ ID NO: 20 - DNA, 31 bp, Artificial Sequence
  Primer
ctgaagcttatgcagaaatacgagaaactgg

SEQ ID NO: 21 - DNA, 30 bp, Artificial Sequence
  Primer
gatgtcgactagggcggacagaagtcggag

(19) United States
(12) Patent Application Publication
(10) Pub. No.: US 2007/0117792 A1
(43) Pub. Date: May 24, 2007

(54) PHARMACEUTICAL COMPOSITIONS FOR SUPPRESSING B-AMYLOID PRODUCTION

(75) Inventors: Toru Watanabe, Tsukuba-shi, Ibaraki (JP); Shigeki Kawabata, Tsukuba-shi, Ibaraki (JP); Shunichiro Hachiya, Tsukuba-shi, Ibaraki (JP); Toshihara Suzuki, Chiba-shi, Chiba (JP)

Correspondence Address: SUGHRUE-265550, 2100 PENNSYLVANIA AVE. NW, WASHINGTON, DC, 20037-3213, US

(73) Assignees: Toshiharu Suzuki, Chiba-shi (JP); ASTELLAS PHARMA INC., Tokyo (JP)

(21) Appl. No.: 11/615,688

(22) Filed: Dec. 22, 2006

Related U.S. Application Data
(62) Division of application No. 10/169,580, filed on Jul. 5, 2002.

(30) Foreign Application Priority Data
Apr. 28, 2000 (JP) P.2000-131037

(51) Int. Cl. A61K 31/55; A61K 31/522

(52) U.S. Cl. 514/215; 514/263.38

(57) ABSTRACT

To provide pharmaceutical compositions for suppressing β-amyloid production on the basis of the cyclin-dependent kinase inhibitory activity. When a substance having a cyclin-dependent kinase inhibitory activity was allowed to contact with nerve cells, the β-amyloid production was suppressed.

### TECHNICAL FIELD

This invention relates to medicaments, particularly a pharmaceutical composition for suppressing β-amyloid (Aβ) production which contains a substance having the cyclin-dependent kinase (Cdk) inhibitory activity as the active ingredient, a method for detecting Aβ production by allowing the substance having Cdk inhibitory activity to contact with cells, and a method for treating dementia or Alzheimer's disease (AD) which comprises administering the pharmaceutical composition for suppressing Aβ production.

### BACKGROUND OF THE INVENTION

Dementia is becoming a serious problem in an aging society. Dementia is a condition under which daily life and social life cannot be sufficiently conducted in various mental functions (e.g., memory, judgement, comprehension, language, capacity, cognition, feeling, volition, character and the like) due to cerebral organic disorders. Among them, AD is characterized by pathological changes such as falling off of nerve cells, reduction of synapse numbers, senile plaque in the brain, accumulation of neurofibrillary change and the like (Science (1990) 248, 1058-1060, N. Engl. J. Med. (1986) 314, 964-973 and Neurobiol. Aging (1995) 16, 365-380). The amyloid substance of senile plaque is a protein called Aβ which is formed when amyloid precursor protein (APP) undergoes metabolism (Nature (1987) 325, 733-736). Two metabolic pathways are known regarding the metabolism of APP, namely a pathway in which it undergoes cutting by α-secretase at the center of the Aβ domain (amyloid non-producing pathway) and a pathway in which it produces Aβ by undergoing Aβ domain preservative cutting by β-secretase and further undergoing cutting by γ-secretase (amyloid producing pathway) (Racchi, M. (1999) Trends Pharmacol. Sci. 20, 418-423, Sinha, S. (1999) Proc. Natl. Acad. Sci. USA 96, 11049-11053). It is known that the ratio of APP metabolized by these two pathways, namely the amount of Aβ production, is changed by the influence of an exogenous or endogenous substance. For example, it is known that ratio of the amyloid non-producing pathway is increased by indirectly changing the metabolism of APP through the acceleration of phosphorylation of molecules other than APP caused by the activation of protein kinase C (PKC) (Racchi, M. (1999) Trends Pharmacol. Sci. 20, 418-423).

In recent years, it has been revealed that formation of Aβ and its accumulation in the brain are deeply concerned in the onset mechanism of AD. Its main research backgrounds are (1) Aβ induces nerve cell death in primary culture nerve cells, (2) it is considered that accumulation of senile plaque is an initial stage change of the pathological change in AD and (3) when mutation type APP gene or mutation type Presenilin 1 or 2 gene which can be found in familial AD is introduced into a host cell, acceleration of Aβ production or increase in the production ratio of Aβ₁₋₄₂ which has higher amyloid formation for Aβ₁₋₄₀ (J. Biol. Chem. (1996) 271, 18295-18298) are observed in comparison with the case of the introduction of wild type APP gene or wild type Presenilin 1 or 2 gene (J. Biol. Chem. (1996) 271, 18295-18298, Science (1997) 275, 630-631 and Proc. Natl. Acad. Sci. USA (1999) 96, 11049-11053).

Also, it is known that APP is phosphorylated in its intracellular domain (Mol. Medicine (1997) 3, 111-123). It has been shown that a species of Cdk, Cdc2 (Cdk1), is concerned in the phosphorylation of Thr668 of APP (the 668th position threonine residue of APP, the amino acid number is based on APP₆₉₅: APP (Thr668) hereinafter) in peripheral cells (non-nerve cells) (EMBO J. (1994) 13, 1114-1122). In recent years, it has been revealed that this residue is phosphorylated also in nerve cell or neuroblastoma such as PC12 cell and that the phosphorylation of this residue takes an important role in the function expression of APP in nerves (J. Neurosci. (1999) 19, 4421-4427).

The aforementioned Cdk has been discovered as serine/threonine kinase which controls cell cycle of eucaryote. Cdk does not show its activity by itself and requires binding with a control subunit called cyclin for its activation. In mammals, the presence of at least 8 species of Cdk have so far been known (Protein, Nucleic Acid and Enzyme (1997) 42, 1554-1561). Cdk5 has been discovered as a member of Cdk due to similarity of its primary structure (EMBO J. (1992) 11: 2909-2917), and it is known that it does not require cyclin for its activation but requires binding with subunits called p35 and p39 instead (Nature (1994) 371 (6496): 423-426, and J. Biol. Chem. (1995) 270: 26897-26903). These subunits are specifically expressed in nerve cells, and it is known that Cdk5 is concerned in functions of nerve cells, namely process elongation reaction in nerve cells and migration of nerve cells during the development process (J. Neurosci. (1999) 19, 6017-6026, Genes & Dev. (1996) 10, 816-825). That is, Cdk5 is not concerned in the control of cell cycle and its function is different from other Cdk though classified as Cdk. As the main substrates of Cdk5, APP, tau, neurofilament and the like are known (J. Neurochem. (2000) 75, 1085-1091, Brain Res. (1997) 765, 259-266, J. Biol. Chem. (1996) 271, 14245-14251, and FEBS Lett. (1993) 336, 417-424).

A large number of Cdk inhibiting substances are known as those which are concerned in the cell growth control, and there are reports stating that they are useful in neurodegenerative disease because of the action mechanism. In addition, these reports describe that said substances may be useful in AD by suppressing formation of a neurofibrillary change having phosphorylated tau as a composing element, based on an assumption that Cdk5 is concerned in the phosphorylation of tau (International Publication WO 99/07705, WO 99/02162, WO 99/65884, WO 99/30710, WO 99/62882 or WO 00/21550, and J. Biochem. (1995) 117, 741-749).

As described above, there are some reports on the relationship between Cdk, particularly Cdk5, and nerve cells and Aβ and AD and on the phosphorylation of APP by Cdk5, but there are no reports which suggest influence of Cdk on APP metabolism, particularly on the Aβ production. That is, there are no reports which suggest influence of a substance having Cdk inhibitory activity on the Aβ production.

### DISCLOSURE OF THE INVENTION

As a result of intensive studies on the elucidation of the mechanism of dementia, particularly AD and the like, the present inventors have found that a substance having Cdk inhibitory activity can suppress Aβ production in nerve cells. Illustratively, as a result of comparative examination on the amount of Aβ production in the presence or absence of said Cdk inhibition substance in a system in which human APP gene is over-expressed in rat primary culture nerve cells, it was revealed that the substance having Cdk inhibitory activity reduces Aβ production. As a result of the achievement of Aβ production suppression, this invention is useful, for example, for the treatment of dementia, Alzheimer's disease and the like.

That is, the invention is a pharmaceutical composition for suppressing β-amyloid production which contains a substance having a cyclin-dependent kinase inhibitory activity as the active ingredient, preferably a pharmaceutical composition for suppressing β-amyloid production which contains a substance having a cyclin-dependent kinase 5 inhibitory activity as the active ingredient.

Also, the invention is a pharmaceutical composition for suppressing β-amyloid production, which is an anti-dementia drug or an anti-Alzheimer's disease drug.

Further, the invention is a pharmaceutical composition for suppressing β-amyloid production, which comprises a substance having an amyloid precursor protein threonine binding phosphorylation inhibitory activity as the active ingredient.

As another embodiment, the invention is a method for detecting β-amyloid production by allowing a substance having cyclin-dependent kinase inhibitory activity to contact with cells, preferably a method for detecting β-amyloid production wherein the cyclin-dependent kinase is cyclin-dependent kinase 5. Also, it is the above method for detecting β-amyloid production, which is a method for screening a β-amyloid production suppresser, and a kit for diagnosing dementia or Alzheimer's disease, which uses the method for detecting β-amyloid production. Also, the invention is a method for detecting β-amyloid production by allowing a substance having an amyloid precursor protein threonine binding phosphorylation inhibitory activity to contact with cells.

### BRIEF DESCRIPTION OF THE DRAWINGS

FIG. 1 shows a result of analysis of total APP amount and degree of phosphorylation of APP (Thr668) by western blotting respectively using 22C11 antibody or phosphorylated APP (Thr668) antibody.

FIG. 2 shows calibration curves of Aβ₁₋₄₀ and Aβ₁₋₄₂ prepared using an Aβ solution having a concentration of from 0.2 to 10 ng/ml.

FIG. 3 shows degree of Aβ₁₋₄₀ production in the case of an Alsterpaullone treatment.

FIG. 4 shows degree of Aβ₁₋₄₂ production in the case of an Alsterpaullone treatment.

FIG. 5 shows degree of Aβ₁₋₄₀ production in the case of a Roscovitine treatment.

### BEST MODE FOR CARRYING OUT THE INVENTION

The following describes the invention in detail.

The “substance having cyclin-dependent kinase (Cdk) inhibitory activity” is a substance which keeps a serine/threonine kinase inhibition function that controls cell cycle of eucaryote or nerve cell functions such as process elongation reaction of nerves and migration of nerve cells during the development process, and which does not particularly require selectivity among Cdk subtypes but keeps inhibitory function at least against Cdk5 which is a Cdk subtype that functions in nerve cells. Examples of such compounds include those which are described in International Publication WO 97/0842, WO 97/16447, WO 98/33798, WO 98/50356, WO 99/07705, WO 99/02162, WO 99/09030, WO 99/15500, WO 99/030710, WO 99/34018, WO 99/62503, WO 99/65910, WO 00/01699, WO 00/12496, WO 00/21926, WO 00/18734, Eur. J. Biochem. (2000), 224, 771, Eur. J. Biochem. (2000), 267, 5983 and Bioorganic & Med. Chem. (1999), 7, 1281 and the like. Their typical examples include Roscovitine, Alsterpaullone or chemically modified products thereof (J. Med. Chem. (1999) 42, 2909-2919, and Trends in Cell Biology (1996) 6, 393-397). Also can be exemplified are commercial products or known compounds registered in Chemical File, compounds obtained by combinatorial chemistry techniques, culture supernatants of microorganisms, natural components derived from plants and marine organisms and animal tissue extracts, or antibodies and dominant negative mutant proteins. In addition, said substances whose substituents and the like are modified by chemical conversion which is a usual method for those skilled in the art can also be exemplified.

Illustratively, it is the aforementioned pharmaceutical composition for suppressing Aβ production which is a compound selected from a condensed heterocyclic ring derivative (I) represented by the following general formula or a pharmaceutically acceptable salt thereof, and Alsterpaullone (II) or a pharmaceutically acceptable salt thereof, 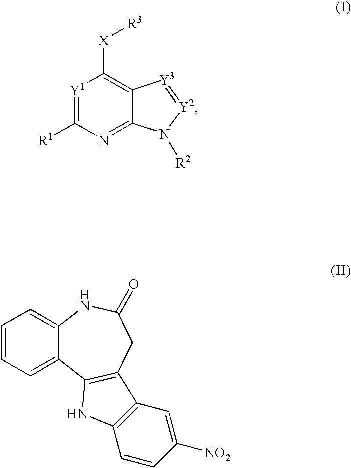
(R¹: H, halogen, OH, SH, hydrocarbon radical-S—, hydrocarbon radical-O—, NR⁴R⁵, hydrocarbon radical which may be substituted, heteroaryl which may be substituted or heterocyclic ring group which may be substituted
X: O, S, S(O)m, CH or NR⁶
m: 1 or 2
R², R³, R⁴, R⁵ and R⁶: H, OH, SH, hydrocarbon radical-S—, hydrocarbon radical-O—, hydrocarbon radical which may be substituted, hydrocarbon radical-O— which may be substituted, heteroaryl which may be substituted or heterocyclic ring group which may be substituted, which may be the same or different from one another
Y¹: N or CR⁷
Y²: N or CR⁸
Y³: N or CR⁹
R⁷, R⁸ and R⁹: H, OH, SH, heteroaryl which may be substituted, hydrocarbon radical which may be substituted, hydrocarbon radical-O— which may be substituted, hydrocarbon radical-S— which may be substituted, hydrocarbon radical-CO— which may be substituted, hydrocarbon radical-O—CO— which may be substituted or R¹⁰R¹¹NCO—, which may be the same or different from one another
R¹⁰ and R¹¹: H, hydrocarbon radical which may be substituted or hydrocarbon radical-O— which may be substituted, which may be the same or different from each other), preferably the aforementioned pharmaceutical composition for suppressing Aβ production which is a purine derivative (III) represented by the following general formula or a salt thereof, 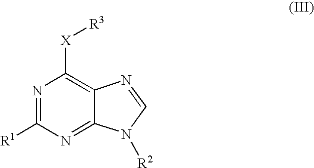
(R¹: H, OH, SH, hydrocarbon radical-O—, hydrocarbon radical-S— which may be substituted, NR⁴R⁵, hydrocarbon radical which may be substituted, heteroaryl which may be substituted or heterocyclic ring group which may be substituted
X: O, S, S(O)m, CH or NR⁶
m: 1 or 2
R², R³, R⁴, R⁵ and R⁶: H, OH, hydrocarbon radical which may be substituted, hydrocarbon radical-O— which may be substituted, heteroaryl which may be substituted or heterocyclic ring group which may be substituted, which may be the same or different from one another), more preferably the aforementioned pharmaceutical composition for suppressing Aβ production which is a 1-aminopurine derivative represented by the following general formula or a salt thereof, 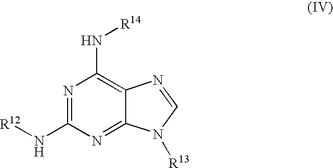
(R¹²: lower alkyl which may be substituted by OH or phenyl, or cycloalkyl or aryl which may be substituted by amino or OH
R¹³: lower alkyl or cycloalkyl
R¹⁴: lower alkyl, lower alkyl-O-lower alkyl or aryl or aralkyl which may be substituted by halogen, OH or lower alkyl-O—).
   With the proviso that, even in the case of said substances, substances having non-selective influence on cells not originated from the Cdk inhibitory activity are excluded. Its example is a case in which said substances have an action to increase the Aβ amount for example by jointly having PKC inhibitory activity.

Symbols in the above general formulae are as follows.

As the “halogen”, fluorine, chlorine, bromine, iodine or the like can be exemplified.

The “hydrocarbon radical” is a group of C₁₋₁₅, preferably C₁₋₁₀ straight or branched chain composed of carbon and hydrogen, and it illustratively means alkyl, alkenyl, alkynyl, cycloalkyl, cycloalkenyl, aryl, cycloalkyl-alkyl, cycloalkenyl-alkyl, aryl-alkyl, cycloalkyl-alkenyl, cycloalkenyl-alkenyl, aryl-alkenyl, cycloalkyl-alkynyl, cycloalkenyl-alkynyl or aryl-alkynyl.

The “alkyl” means a straight or branched saturated hydrocarbon radical, preferably a C₁₋₁₀ alkyl, and its illustrative examples include methyl, ethyl, isopropyl, hexyl, decyl and the like.

The “alkenyl” means a straight or branched hydrocarbon radical having at least one or more double bonds, preferably a C₂₋₁₀ alkenyl, and its illustrative examples include vinyl, propenyl, allyl, isopropenyl, hexenyl and the like.

The “alkynyl” means a straight or branched hydrocarbon radical having at least one or more triple bonds, preferably a C₂₋₁₀ alkynyl, and its illustrative examples include ethynyl, propynyl, butenyl and the like.

The “cycloalkyl” and “cycloalkenyl” mean monocyclic saturated and unsaturated hydrocarbon radicals, preferably a “C₃₋₈ cycloalkyl” and a “C₃₋₈ cycloalkenyl”, and their illustrative examples include cyclopropyl, cyclopentyl, cyclohexyl, cyclopentenyl and the like.

The “aryl” means an aromatic hydrocarbon radical, preferably a C₆₋₁₄ aryl, and its illustrative examples include phenyl, tolyl, cumenyl, xylyl, naphthyl, biphenyl and the like.

The “cycloalkyl-alkyl”, “cycloalkenyl-alkyl” and “aryl-alkyl” are groups in which hydrogen at an optional position of the aforementioned alkyl is substituted by the aforementioned cycloalkyl, cycloalkenyl and aryl, and their illustrative examples include cyclohexylmethyl, benzyl, phenethyl and the like.

The “heteroaryl” is a five- or six-membered monocyclic heteroaryl containing from 1 to 4 hetero atoms selected from N, S and O and a bicyclic heteroaryl condensed with benzene ring, which may be partially saturated. Furyl, thienyl, pyrrolyl, imidazolyl, pyrazolyl, thiazolyl, oxazolyl, pyridyl, pyrazinyl or pyrimidyl can be cited as the monocyclic heteroaryl, and benzofuranyl, benzothienyl, benzothiaziazolyl, benzothiazolyl, benzimidazolyl, indolyl, quinolyl or quinoxalinyl can be cited as the bicyclic heteroaryl. Preferred is a five- or six-membered monocyclic heteroaryl, particularly furyl, thienyl, imidazolyl, thiazolyl and pyridyl.

The “heterocyclic ring group” means a three- to seven-membered saturated or non-aromatic unsaturated ring group containing from 1 to 3 hetero atoms selected from nitrogen atom, oxygen atom or sulfur atom, and its examples include oxazolidinyl, tetrahydrofuranyl, 1,4-dioxanyl and tetrahydropyran.

As the “acyl”, HCO—, C₁₋₂₅ hydrocarbon radical-CO—, C₁₋₂₅ hydrocarbon radical-CS—, heteroaryl-CO—, heteroaryl-alkyl-CO—, heteroaryl-alkenyl-CO—, heteroaryl-alkynyl-CO—, hetero ring-CO—, hetero ring-alkenyl-CO—, HCS—, heteroaryl-CS—, heteroaryl-alkyl-CS—, heteroaryl-alkenyl-CS—, heteroaryl-alkynyl-CS—, hetero ring-CS— or hetero ring-alkenyl-CS— can be cited. It is preferably a C₁₋₂₅ hydrocarbon radical-CO—, and its illustrative examples include formyl, acetyl, propionyl, 2-methylbut-2-enoyl, benzoyl and the like.

The “hydrocarbon radical-O—” means an alkoxy such as methoxy, ethoxy, phenoxy or the like.

The “esterificated carboxyl” means a “hydrocarbon radical-O—CO—” such as methoxycarbonyl, ethoxycarbonyl or the like. Groups substituted by the aforementioned acyl are also included in the esterificated carboxyl.

The substituent in the “hydrocarbon radical which may be substituted”, “hydrocarbon radical-O— which may be substituted”, “heteroaryl which may be substituted” or “heterocyclic group which may be substituted” is not particularly limited with the proviso that it is a group which can be substituted on these rings. These have 1 to 5 optional substituents which may be the same or different from one another.

Illustratively, it is a group selected from the following group A.

Substituent A

Halogen; CN; NO₂; alkoxy; R^{a}R^{b}N— (R^{a} and R^{b}: the same or different from each other and each represents hydrogen atom, hydrocarbon radical, heteroaryl, heteroaryl-alkyl, hetero ring, hetero ring-alkyl or acyl (the same shall apply hereinafter)); hydrocarbon radical which may be substituted by esterificated carboxyl or R^{a}R^{b}N—; heteroaryl which may be substituted by hydrocarbon radical; hetero ring which may be substituted by hydrocarbon radical; acyl;
D¹-G¹- (D¹: heteroaryl, hetero ring or R^{a}R^{b}N—, G¹: —O—, —S(O)ₙ—, —O—CO—, —CO—, —CS—, —O—CO—CO— or —CO—O—, n: 0, 1 or 2);
D²-G²- (D²: H, acyl-C₁₋₆ alkyl, heteroaryl-C₁₋₆ alkyl or hetero ring-C₁₋₆ alkyl, G²: —O—, —S(O)ₙ—, —O—CO—, —CO—, —CS—, —O—CO—CO— or —CO—O—);
D³-G³- (D³: hydrocarbon radical which may be substituted by alkoxy or esterificated carboxy, G³: —S(O)ₙ—, —O—CO—, —O—CO—CO— or —CO—O—).

The active ingredient of the invention has double bond so that it exists in geometrical isomer and tautomer forms. Isolated or mixed form these isomers are included in the invention.

Also, depending on the types of substituents, the compound of the invention may have asymmetric carbon atom so that isomers based on the asymmetric carbon can be present. Mixed or isolated forms of these optical isomers are included in the invention. In addition, compounds obtained by labeling the compound of the invention with radioactive isotopes are also included in the invention.

Further, the active ingredient of the invention may form acid addition salts or salts with bases depending on the type of substituents, and such salts are included in the invention so far as they are pharmaceutically acceptable salts. Their illustrative examples include acid addition salts with inorganic acids (e.g., hydrochloric acid, hydrobromic acid, hydriodic acid, sulfuric acid, nitric acid, phosphoric acid and the like) with organic acids (e.g., formic acid, acetic acid, propionic acid, oxalic acid, malonic acid, succinic acid, fumaric acid, maleic acid, lactic acid, malic acid, tartaric acid, citric acid, methanesulfonic acid, ethanesulfonic acid, p-toluenesulfonic acid, aspartic acid, glutamic acid and the like), salts with inorganic bases (e.g., sodium, potassium, magnesium, calcium, aluminum and the like) and with organic bases (e.g., methylamine, ethylamine, ethanolamine, lysine, ornithine and the like), and ammonium salts and the like. Further, said substance may sometimes be isolated as hydrates, various types of solvates (e.g., ethanol solvate and the like) or as their polymorphic substances, and these various hydrates, solvates and polymorphic substances are also included in the invention.

Also, all of the compounds so called prodrugs which are metabolized and converted in the living body are included in the active ingredient of the invention. As the group which forms the prodrug of the invention, the groups described in Prog. Med., 5, 2157-2161 (1985) and “Development of Medicaments” Vol. 7 (Hirokawa Shoten, 1990) Molecular Designing 163-198 and the like can be exemplified.

The “β-amyloid (Aβ)” is an amyloid substance which forms senile plaque. Said substance is also called β protein and formed by the degradation of APP by protease (processing) (Nature, (1987) 325, 733-736). Illustratively, Aβ means a fragment formed from APP during its intracellular secretion process after biosynthesis or a process in which it is incorporated from cell membrane and undergoes metabolism, by undergoing cutting by protease (processing), cutting by β-secretase Aβ domain conservatively and further undergoing cutting by γ-secretase (Trends Pharmacol. Sci. 20, 418-423, and Proc. Natl. Acad. Sci. USA (1999) 96, 11049-11053). This is mainly a protein called Aβ composed of 40 or 42 amino acid residues, namely Aβ₁₋₄₀ (SEQ ID NO;1) or Aβ₁₋₄₂ (SEQ ID NO;2), but other than this, variants thereof in which one or two or more amino acids are substituted, deleted or inserted can also be exemplified.

The “amyloid precursor protein: APP” means a precursor of the aforementioned Aβ and is a one transmembrane type membrane protein preferably having a short cytoplasmic domain composed of 47 amino acids or a domain in which one or two or more amino acids of said cytoplasmic domain may be substituted, deleted or inserted. It illustratively means each of three protein isoforms APP₆₉₅ (SEQ ID NO;3) , APP₇₅₁ (SEQ ID NO;4) and APP₇₇₀ (SEQ ID NO;5), respectively composed of 695, 751 and 770 amino acids.

The phosphorylation of APP″ means addition of phosphoric acid to APP, which occurs when APP undergoes metabolism in the living body for example during its intracellular secretion process, on the cell membrane or after its incorporation from the cell membrane. Though illustrative addition position of phosphoric acid to APP depends on the length of APP, in the case of APP₆₉₅, it means that the 668th threonine residue positioned in said APP intracellular domain (APP(Thr668)) (the amino acid number is based on APP₆₉₅) is phosphorylated (Mol. Medicine (1997) 3, 111-123).

The “substance having APP phosphorylation inhibitory activity” is a substance which inhibits phosphorylation of the 668th threonine residue positioned in the APP intracellular domain. Though it may be any substance which has said inhibitory activity, its illustrative examples include commercial products or known compounds registered in Chemical File, compounds obtained by combinatorial chemistry techniques, culture supernatants of microorganisms, natural components derived from plants and marine organisms and animal tissue extracts, or antibodies and dominant negative mutant proteins. In addition, said substances whose substituents and the like are modified by chemical conversion which is a usual method for those skilled in the art can also be exemplified.

The “dominant negative mutant” is a substance capable of specifically inhibiting activity of a protein by its competition with the binding of the protein with its substrate, a regulatory factor or the like, using a protein whose activity is deleted by introducing mutation into active site or activity regulating site of the protein, illustratively in the case of Cdk5, those in which the 144th asparagine residue as an important site for phosphoric acid transfer reaction was mutated into aspartic acid residue and the 33rd threonine residue as the ATP binding site was mutated into lysine residue are known as dominant negative mutant proteins (Genes & Development 10, 816-825 (1996)).

The “dementia” means a condition under which daily life and social life cannot be sufficiently made in various mental functions (e.g., memory, judgement, comprehension, language, capacity, cognition, feeling, volition, character and the like) due to cerebral organic disorders.

Though the “Alzheimer's disease (AD)” is sometimes strictly classified into AD which occurs at the middle-aged stage and senile dementia of type which occurs after 65 years or more of age, both cases are jointly called AD in this specification.

Accordingly, the “anti-dementia drug” or “anti-Alzheimer's disease drug” means a medicament for healing or improving the aforementioned diseases.

The “method for detecting β-amyloid production by allowing a substance having cyclin-dependent kinase (Cdk) inhibitory activity to contact with cells” or “method for detecting β-amyloid production by allowing a substance having an action to inhibit phosphorylation of threonine binding phosphoric acid of amyloid precursor protein to contact with cells” are described in detail in the following, and their examples include a kit for screening or diagnosing said substances and a test method for elucidating mechanism of AD. Preferred is a screening method of said substances.

The method of the invention for detecting Aβ production is described in detail.

Preparation of Vector DNA Designed for Expressing APP Gene:

Structures of APP genes are preserved broadly from mammals to insects (Proc. Natl. Acad. Sci. USA (1992) 89, 10758-10762, Proc. Natl. Acad. Sci. USA (1989) 86, 2478-2482, Biochem. J. (1998) 330, 29-33, and Proc. Natl. Acad. Sci. USA (1993) 90, 12045-12049), and one or two or more APP genes of any APP species by which the invention can be carried out or mutants thereof in which amino acids are substituted, deleted or inserted are also included. Also included therein are mutation type APP found in familial AD (Nature (1991) 349, 704-706, Science (1991) 254, 97-99, Nature (1991) 353, 844-846, Lancet (1991) 337, 978-979, Biochem. Biophys. Res. Comm. (1991) 178, 1141-1146, Lancet (1991) 337, 1342-1343, and Nature Genet. (1992) 1, 343-347). Also, APP₆₉₅, APP₇₅₁ and APP₇₇₀ having different sequences due to difference in splicing are produced from the APP genes (Biotechnology (1989) 7, 147-163), and the APP as used herein includes all of these proteins produced from the APP genes.

As the vector DNA, any of known gene transfer vectors which do not exert influence for carrying out the invention can be used, including virus-derived vectors such as adenovirus vectors and the like (Proc. Natl. Acad. Sci. USA (1998) 95, 2509-2514) and expression vectors for mammal cells such as pEF-BOS (Nucleic Acids Res. (1990) 18, 5322), pSSR α (Mol. Cell. Biol. (1988) 8, 466-472) and the like. The promoter to be used in the expression of APP is not particularly limited, too.

Cells and Animals to be Used in the Test:

A nerve cell primary culture system can be used as the host cell. Preferably, the primary culture nerve cells are animal-derived cells, particularly mammal cells. For example, they are cells of a mammal generally used as an experimental animal, preferably rat or mouse, and preparation of the culture system is carried out in accordance with a known method (Dev. Brain. Res. (1986) 30, 47-56). It is desirable that the culturing period of the nerve cell primary culture system is the 7th day or more when the cells are sufficiently differentiated, preferably from the 7th to 10th day. A primary culture from an animal treated with gene manipulation is also included in said animal cells. For example, a cell strain established by separating from a temperature sensitive oncogene introduced transgenic mouse prepared by Taitoh et al. (a transgenic mouse expressing SV40 temperature sensitive T antigen by the SV40 self promoter (Exp. Cell Res. (1991) 197, 50-56) can also be exemplified as the host cell. Also can be exemplified are PC12 cell strain (J. Cell. Biol. (1978) 78, 747-755) and the like already established culture cell strains. The cells can be used are not particularly limited to nerve derived cells. In addition, cells of other than mammals can also be used, for example, Drosophila derived cells can be cited.

The APP gene can also be expressed transiently or stably in cells by using the calcium phosphate method (Virology (1973) 52, 456-467), Lipofectamine method (LIFE TECHNOLOGIES), FUGENE™ 6 method (BOEHRINGER MANNHEIM) and the like as the gene transfer method.

An expression vector designed such that expression of APP can be induced by a certain stimulation can also be expressed stably in cells. For example, in the case of the LacSwitch method (Nucleic Acids Res. (1991) 191, 4647-4653), expression of APP protein can be induced by adding IPTG (isopropyl-beta-D-thiogalactoside) to a medium. When expressed amount of endogenous APP is sufficient for measuring the amount of produced Aβ, it is possible to carry out the invention without particularly over-expressing exogenous APP.

In addition, an APP gene-introduced transgenic animal can also be used. Transgenic animals can be prepared by injecting an isolated gene into fertilized eggs and then transplanting the fertilized eggs into a false pregnancy animal to allow them to develop into individuals (Science (1981) 214, 1244-1246). In this case, Aβ production suppressing action of a compound to be tested can be observed at individual level. These transgenic animals can also be used for the detection of Aβ production suppressing action of a compound to be tested, by crossing them with a presenilin or Cdk5 over-expressing transgenic animal. In addition, the APP gene can also be introduced directly into an animal individual by a virus vector or the like. Those derived from viruses such as herpes simplex virus, adenovirus and the like can be used as the virus vector (J. Neurosci. (1996) 16, 486-496 and J. Neurosci. Methods. (1997) 71 77-84).

Measurement of Aβ Production:

It is known that Aβ is formed and released outside the cells even under normal conditions (Nature (1992) 359, 322-325, and Science (1992) 258, 126-129), and extracellular accumulation of Aβ is found also in the brain of AD. Accordingly, in the case of culture cells, it is desirable to carry out the Aβ production measuring method by measuring the amount of Aβ released in the culture medium to be used as the Aβ production. Though indirect, it is possible to measure the amount of Aβ which is present in the cells. In addition, in the case of a test using animal individuals including human, it is possible to measure the amount of Aβ in body fluids such as cerebrospinal fluid and the like and whole tissues. Accordingly, the detection method of the invention can be used in a diagnosing kit which measures the amount of Aβ by administering the pharmaceutical composition of the invention to patients.

Enzyme-linked immunosorbent assay (ELISA) and the like known methods are used for the measurement of the amount of Aβ. In the enzyme-linked immunosorbent assay, for example, an anti-Aβ antibody such as 6E10 (Wako) or the like is allowed to react with Aβ]in a collected cell culture medium or Aβ₁₋₄₀ or Aβ₁₋₄₂ to be used as the standard, as the first step, this is allowed to react with an antibody such as R163, R165 (DR. P D Mehta of New York State Institute for Basic Research) or the like respectively capable of specifically binding to Aβ₁₋₄₀ or Aβ₁₋₄₂, as the second step, and then an appropriate anti-IgG antibody labeled with an enzyme (e.g., peroxidase or the like) is allowed to react therewith. The amount of Aβ in a culture medium is determined by measuring the activity of the enzyme (e.g., peroxidase or the like) (Biochem. Biophys. Res. Commun. (1972) 47, 846-851). Signal Select™ (BIOSOURCE), Human amyloid β (1-42) Measuring Kit (IBL) or the like commercially available kit can also be used in the ELISA method. In addition, determination methods by western blotting, dot blotting and the like can also be used (Nature (1970) 227, 680-685, and Proc. Natl. Acad. Sci. USA (1979) 76, 4350-4354).

Evaluation of Compounds to be Tested:

By the above methods, a method for the detection of Aβ production suppressing action based on Cdk, preferably Cdk5, inhibitory activity using a large number of compounds to be tested, or a method for the detection of Aβ production suppressing action based on the inhibition of APP threonine phosphate phosphorylation, can be cited. For example, a compound to be tested is added to a cell culture medium to a final concentration of 1 nM to 200 μM, and after a predetermined period of time, the culture medium is collected to determine the produced amount of Aβ by the aforementioned method. The Cdk5 inhibitory activity can be determined by allowing Cdk5 purified from an animal cell or animal tissue by immunoprecipitation or using an appropriate column or recombinant Cdk5 or p25 expressed in and purified from Sf9 cell or the like insect cell (p35 or p39 or respective active site fragment thereof, p21, N145, p30 or the like can be used (Nature (1994) 371 (6496): 423-426, J. Biol. Chem. (1997) 272: 12318-12327, and J. Biol. Chem. (1995) 270: 26897-26903) to react with isotope-labeled ATP and an appropriate protein or peptide to be used as the substrate in an appropriate buffer, and then measuring amount of the isotope incorporated into the substrate by so-called scintillation proximity kinase assay (SPA method) or autoradiography (Eur. J. Biochem. (1997) 243, 527-536, and Biochem. (1999) 268, 318-329). The degree of APP phosphorylation can be determined after solubilization of cells using a solubilizing agent, for example by western blotting method using an antibody which specifically react with phosphorylated state of APP (J. Neurosci. (1999) 19, 4421-4427).

As the compounds to be tested which can be evaluated by the method of the invention, commercial products or known compounds registered in Chemical File and compounds obtained by combinatorial chemistry techniques can be used. In addition, culture supernatants of microorganisms, natural components derived from plants and marine organisms, animal tissue extracts and the like can also be used. Also, antibodies, dominant negative mutant proteins and the like can be used, too. Also useful are chemically modified products of substances found by the method of the invention.

The following describes the pharmaceutical composition of the invention for suppressing Aβ production in detail.

The pharmaceutical composition of the invention for suppressing Aβ production is prepared into tablets, powders, fine subtilaes, granules, capsules, pills, solutions, injections, suppositories, ointments, adhesive preparations and the like using generally used pharmaceutical carriers, fillers and other additives and administered orally (including sublingual administration) or parenterally.

Clinical dose of the pharmaceutical composition of the invention for suppressing Aβ production in human is optionally decided by taking into consideration symptoms, weight, age, sex, route of administration and the like of each patient to be treated, but is usually within the range of from 0.1 mg to 5,000 mg, preferably from 1 mg to 500 mg, per day per adult by oral administration, once a day or dividing the daily dose into several doses, or within the range of from 0.1 mg to 5,000 mg, preferably from 1 mg to 500 mg, per day per adult by parenteral administration, once a day or dividing the daily dose into several doses, or by intravenous continuous administration within the range of from 1 hour to 24 hours per day. As a matter of course, since the dose varies under various conditions as described in the foregoing, a smaller dose than the above range may be sufficient enough in some cases.

As the dosage form of the pharmaceutical composition of the invention for suppressing Aβ production, tablets, powders, granules and the like are used. In such dosage forms, one or more active substances are mixed with at least one inert diluent such as lactose, mannitol, glucose, hydroxypropylcellulose, microcrystalline cellulose, starch, polyvinyl pyrrolidone or aluminum magnesium silicate. In the usual way, the composition may contain other additives than the inert diluent, such as a lubricant (e.g., magnesium stearate or the like), a disintegrating agent (e.g., calcium cellulose glycolate or the like), a stabilizing agent (e.g., lactose or the like) and a solubilization assisting agent (e.g., glutamic acid, aspartic acid or the like). If necessary, tablets or pills may be coated with a film of a gastric or enteric substance such as sucrose, gelatin, hydroxypropylcellulose, hydroxypropylmethylcellulose phthalate or the like. Said dosage forms further include pharmaceutically acceptable emulsions, solutions, suspensions, syrups, elixirs and the like, and contain a generally used inert diluent such as purified water or ethanol. In addition to the inert diluent, this composition may also contain auxiliary agents such as a solubilizing or solubilization assisting agent, a moistening agent, a suspending agent and the like, as well as sweeteners, flavors, aromatics and antiseptics.

The injections for parenteral administration includes aseptic aqueous or non-aqueous solutions, suspensions and emulsions. Examples of the diluent for use in the aqueous solutions and suspensions include distilled water for injection and physiological saline. Examples of the diluent for use in the non-aqueous solutions and suspensions include propylene glycol, polyethylene glycol, plant oils (e.g., olive oil or the like), alcohols (e.g., ethanol or the like), polysorbate 80 (trade name) and the like. Such a composition may further contain additive agents such as a tonicity agent, an antiseptic, a moistening agent, an emulsifying agent, a dispersing agent, a stabilizing agent (e.g., lactose) and a solubilizing or solubilization assisting agent. These compositions are sterilized by filtration through a bacteria retaining filter, blending of a germicide or irradiation. Alternatively, they may be used by firstly making into sterile solid compositions and dissolving them in sterile water or a sterile solvent for injection prior to their use.

In order to disclose the invention further illustratively, its examples are described in the following, though the invention is not limited the examples.

### EXAMPLE 1

Quantity of APP Phosphorylation in Rat Hippocampus Primary Culture Nerve Cells and Construction of Aβ Production Measuring System:

(1) Preparation of APP Adenovirus Vector:

A human APP₆₉₅ gene encoding 695 amino acids (SEQ ID NO;6) was used as the APP gene. An adenovirus vector was prepared in accordance with the ADEasy system (Proc. Natl. acad. Sci. USA (1998) 95, 2509). In order to add CAG promoter, after blunt-end treatment of termini, the APP₆₉₅ gene was once introduced into HincII site of pCAG-pA (Gene (1991) 108, 193-200), and then the CAG promoter and its downstream APP gene were cut out using PstI and XhoI sites, blunt-ended and then introduced into the EcoRV site of a shuttle vector, pADTrack, of the ADEasy system. The APP gene-introduced shuttle vector was converted into linear strand using PmeI and introduced into an Escherichia coli strain BJ5183 together with pADEasy-2 vector to prepare a viral genome recombinant plasmid.

(2) Preparation of Adenovirus:

The APP gene-containing recombinant adenovirus vector was converted into linear strand by PacI treatment and introduced into HEK293 cells by the Lipofectamine method (LIFE TECHNOLOGIES), and 3 to 5 days thereafter when cells were pealed off from the plate, all of the cells were recovered. The recombinant adenovirus was obtained from the supernatant by repeating freezing-thawing step of the cells. The thus obtained recombinant adenovirus was prepared in a large amount by repeating infection of HEK293 cells and then concentrated and purified by the CsCl method and used in the test.

(3) Preparation of Rat Primary Culture Nerve Cells:

A pregnant rat was anesthetized with diethyl ether and allowed to cause death due to hemorrhage by thoracic (heart) incision, and then the womb was extracted by incising the abdominal side. The fetus was sterilized with ethanol for disinfection in a clean bench, the total brain was extracted and recovered in a dish containing a serum-free medium (SUMILON, Sumitomo Bakelite), and the hippocampus was extracted using a precision pincette under a stereoscopic microscope. The hippocampus was transferred into a 50 ml tube, tissues were precipitated by standing to discard the supernatant by aspirating and a cell dispersing solution (PBS containing 1% papain, 150 U/ml DNase I, 0.02% L-cysteine, 0.02% BSA and 0.5% glucose) was added thereto, and the mixture was incubated at 37° C. for 15 minutes and then centrifuged (1,000 rpm, 4° C., 5 min). The supernatant was removed by aspiration, 10 ml of the serum-free medium was added to the sediment, pipette treatment was carried out several times, and then cell masses were removed using a filter to obtain a cell suspension. The number of cells was counted using trypan blue, and 1.5×10⁵ of the cells were inoculated into a poly-L-lysine-coated 48 well culture plate. In order to effect sufficient differentiation, they were cultured at 37° C. for 10 days in a CO₂ incubator and then used in the test.

(4) Infection of Primary Culture Nerve Cells with Adenovirus:

Infection with adenovirus was carried out on the 10th day of the culturing. A virus suspension of 1.5×10⁸ particles was suitably diluted and added to a medium to an MOI (multiplicity of infection) value of 1,000 based on 1.5×10⁵ cells. A sample was recovered on the 4th day of the infection.

(5) Measurement of APP Phosphorylation:

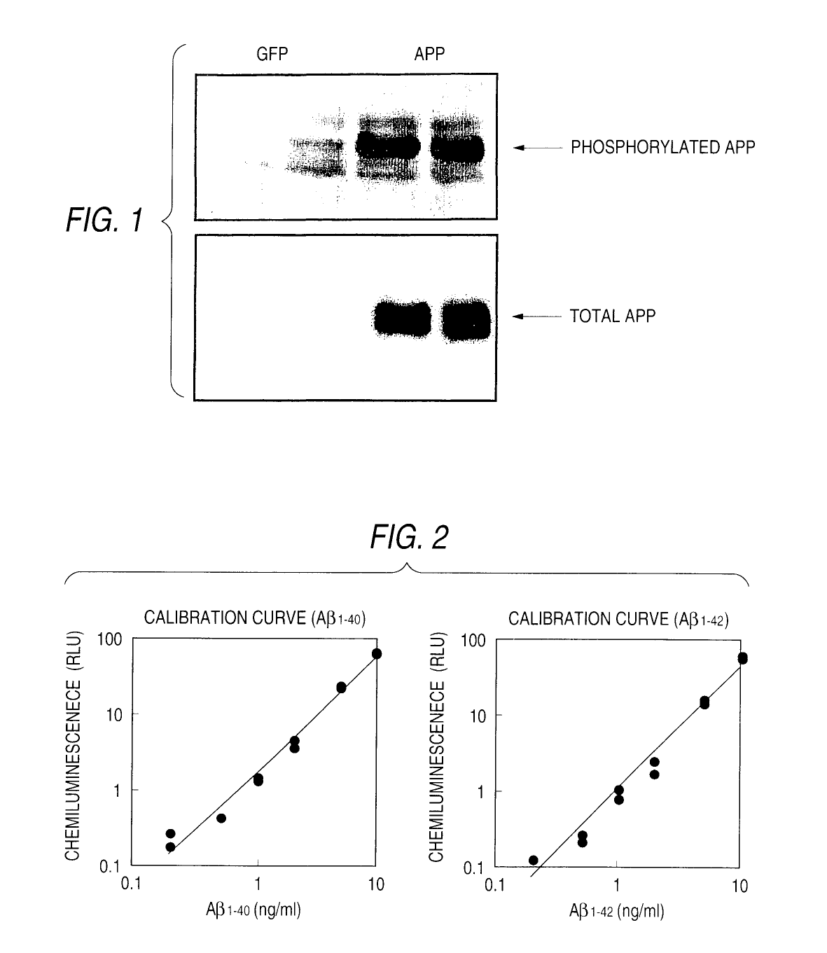

A 50 μl portion of cell lysis solution (50 mM Tris-HCl, 1% SDS, 2.7 M urea, 1 mM Na₃VO₄, 1 mM NaF) was added to 1.5×10⁵ cells, the mixture was recovered in a tube, subjected to about 5 seconds of ultrasonic disintegration and then centrifuged (15,000 rpm, 5 min), and the thus obtained supernatant was used as a sample. A portion of the sample was separated by SDS-PAGE (7.5% polyacrylamide gel) and then transferred onto a PVDF (polyvinylidene difluoride) membrane (Daiichi Pure Chemicals) using a semi-dry blotting apparatus (BIO-RAD). The thus obtained PVDF membrane was soaked in Block Ace (Daiichi Pure Chemicals) at 4° C. overnight to carry out blocking. A polyclonal antibody (anti-phosphorylation APP(Thr668) antibody) for use in the detection of the phosphorylation of APP(Thr668) was prepared by preparing a peptide in which phosphate group was introduced into the T residue of an amino acid sequence NH₂-AAVTPEERHC (SEQ ID NO;7) corresponding to the intracellular region of APP, binding it to KLH, and then immunizing a rabbit therewith (J. Neurosci., 19, 4421-4427 (1999)). The anti-serum obtained by the immunization was used by subjecting to adsorption treatment using an immunogen peptide before introduction of phosphate group and then purifying by affinity chromatography using the phosphate group-introduced antigen peptide. Also, an anti-APP monoclonal antibody 22C11 (Roche Molecular Biochemicals) was used in the determination of total APP amount (combined APP amount of phosphorylated state of APP and non-phosphorylated state of APP). The PVDF membrane after blocking was lightly washed with PBS-T (PBS (140 mM NaCl, 10 mM phosphate buffer, pH 7.4) containing 0.05% Tween 20), soaked in a 1,000 times diluted primary antibody (anti-phosphorylation APP(Thr668) antibody or 22C11) solution and allowed to undergo the reaction at room temperature for 1 hour. After completion of the reaction, washing with PBS-T at room temperature for 10 minutes was repeated four times and then this was subjected to the reaction with a secondary antibody. As the secondary antibody, 20,000 times diluted HRP-labeled anti-rabbit and anti-mouse IgG antibodies (Amersham Pharmacia Biotech) were used. In the same manner as the case of the primary antibody reaction, they were allowed to undergo the reaction at room temperature for 1 hour and then subjected to washing with PBS-T in the same manner. ECL-plus detection kit (Amersham Pharmacia Biotech) was used in the detection of the reaction, and analysis of chemiluminescence was carried out by Storm860 Imageanalyzer (Amersham Pharmacia Biotech) and autoradiography. FIG. 1 shows a result of analysis of total APP amount and degree of phosphorylation of APP (Thr668) by western blotting respectively using 22C11 antibody or phosphorylated APP (Thr668) antibody, after infection of primary culture nerve cells with wild type APP₆₉₅ adenovirus. As a control, the cells were infected with adenovirus which had been integrated with GFP (jellyfish green fluorescent protein, Neuron (1996) 16, 255-260) (SEQ ID NO;8). Significant increase in the total APP and phosphorylation of APP (Thr668) was observed by the infection with wild type APP₆₉₅ adenovirus.

(6) Measurement of Aβ Production:

Culture supernatants recovered 24 hours after the addition of compounds to be tested were used as the samples for Aβ production measurement. Amount of Aβ in each supernatant was determined by sandwich ELISA. Firstly, an anti-Aβ monoclonal antibody 6E10 (Wako) diluted to 10 μg/ml with 100 mM phosphate buffer was dispensed in 50 μl/well portions into a plate for ELISA (Maxisorp, Nunc) and allowed to stand overnight at 4° C. to effect adhesion of the 6E10 antibody to the plate. The antibody was recovered on the next day, and 200 μl of Block Ace was added to each well to carry out blocking at 4° C. overnight or more. The plate was washed 4 times with TBS-T (TBS (20 mM Tris pH 7.5, 150 mM NaCl) containing 0.05% Tween 20) using a plate washer (Bio-Rad, Model 1250 Immuno Wash), and then a sample diluted 2 times with Block Ace or Aβ for calibration curve preparation was added and allowed to undergo the reaction at 4° C. overnight. The Aβ for calibration curve preparation was prepared in the following manner. Each of Aβ₁₋₄₀ (SEQ ID NO;1) and Aβ₁₋₄₂ (SEQ ID NO;2) was purchased from California Peptide and dissolved in hexafluoro-2-propanol (HFIP, Kanto Kagaku) to a concentration of 1 mg/ml to be used as a stock solution. A 10 μl portion of the stock solution was transferred into a tube, HFIP was evaporated using a vacuum concentrator and then the residue was again dissolved in 10 μl of DMSO to an Aβ concentration of 1 mg/ml. Serial dilutions of the solution for Aβ calibration curve measurement were prepared within the range of from 0.2 to 10 ng/ml. The plate after the reaction with a sample or Aβ for calibration curve preparation was washed 4 times with the plate washer and then allowed to react with primary antibodies. Polyclonal antibodies R163 (Aβ₁₋₄₀ specific antibody) and R165 Aβ₁₋₄₂ specific antibody), which specifically recognize C-terminus of Aβ, were used as the primary antibodies. R163 and R165 were purchased from DR. P D Mehta (New York State Institute for Basic Research) and purified as IgG fraction. Each antibody was diluted 1,000 times with TBS-T containing 25% Block Ace, dispensed in 60 μl/well portions into the plate and then allowed to undergo the reaction at room temperature for 2 hours. After the reaction, the plate was washed 4 times using the plate washer and then the reaction with a secondary antibody was carried out. An HRP-labeled anti-rabbit IgG antibody was used as the secondary antibody, diluted 8,000 times with TBS-T containing 25% Block Ace and dispensed in 60 μl/well portions, and then the reaction was carried out at room temperature for 2 hours. After completion of the reaction, the plate was washed 4 times using the plate washer and then chemiluminescence by a POD chemiluminescence reagent (Boehringer Mannheim) was measured by ML3000 Plate Reader (Dynatech Laboratories). FIG. 2 shows calibration curves of Aβ₁₋₄₀ and Aβ₁₋₄₂ prepared using an Aβ solution adjusted to a concentration of from 0.2 to 10 ng/ml.

### EXAMPLE 2

Measuring Method of Cdk5 Activity and Evaluation of Cdk5 Inhibitory Activity of Alsterpaullone and Roscovitine

(1) Preparation of Cdk5 and p25:

Regarding the active site of Cdk5 gene (EMBO J., 11(8), 2909-2917 (1992)) and p35 gene (Nature, 371(6496), 419-423 (1994)), namely a region corresponding to p25, its human type was obtained by RT-PCR. Complete length of the human Cdk5 gene was amplified from total RNA prepared from a human neuroblastoma SH-SY5Y using a primer set of 5′-TACGGATCCGCAGAAATACGAGAAACTGG-3′ (SEQ ID NO;9) and 5′-CTGAAGGTTTAGGGCGGACAGAAGTCGGAG-3′ (SEQ ID NO;10). The total RNA was obtained by adding 1 ml of ISOGEN reagent (Nippon Gene) to 1×10⁶ cells, mixing the resulting lysate with ⅕ volume of chloroform, centrifuging the mixture at 15,000 rpm for 10 minutes, mixing the resulting supernatant with ½ volume of 2-propanol and further centrifuging the mixture at 15,000 rpm for 20 minutes. The thus obtained RNA was dissolved in water and, after measurement of its concentration, subjected to PCR reaction (Titan™ one tube RT-PCR kit (Boehringer Mannheim)). Also, the active site of human p35 gene, namely a region corresponding to p25, was amplified in the same manner using a primer set of 5′-TACGGATCCCCAGGCGTCCACCAGTGAG-3═ (SEQ ID NO;11) and 5′-TACAAGCTTCATGACGCAGGCTACAGTGC-3′ (SEQ ID NO;12). Each of the genes was introduced into a Bacmid preparation vector pFASTBac-Hta (Gibco BRL) which had been designed such that His-tag is added to N-terminus, making use of the HindIII and BamHI sites designed in the primers. Each of the genes introduced into pFASTBac was introduced into DH10Bac to carry out recombination in E. coli, thereby obtaining respective recombinant Bacmids. The thus obtained Bacmid was introduced into Sf9 cells using cellfectin reagent (Gibco BRL) to obtain baculovirus. By infecting Sf9 cells with the thus obtained baculovirus, recombinant Cdk5 protein (SEQ ID NO;13: the Cdk5-originated sequence is in and after the 28th position amino acid) and p25 protein (SEQ ID NO;14: the p25-originated sequence is in and after the 28th position amino acid) produced by the baculovirus were obtained. Purification of the recombinant proteins was carried out making use of the 6× His tag added to the N-terminus.

That is, the Sf9 cells infected with baculovirus were lysed using a cell lysis solution (50 mM Tris-HCl, 10 mM 2-ME, 1 mM PMSF, 1% NP-40), subjected to ultrasonic disintegration and then centrifuged (10,000×g, 30 min), and the thus obtained supernatant was applied to Ni—NTA column (QIAGEN) which had been equilibrated with a column buffer solution A (20 mM Tris-HCl, 500 mM KCl, 20 mM imidazole, 10 mM 2-ME, 10% glycerol). After though washing with the buffer A, this was washed with a buffer B (20 mM Tris-HCl, 1 M KCl, 10 mM 2-ME, 10% glycerol). Finally, fractions eluted with a buffer C (20 mM Tris-HCl, 100 mM KCl, 100 mM imidazole, 10 mM 2-ME, 10% glycerol) were recovered, dialyzed against a sample solution (50 mM HEPES-KOH, 10 mM MgCl₂, 2.5 mM EGTA, 0.1 mM PMSF) and used as a purified preparation.

(2) Measurement of Cdk5 Activity:

A 1:1 mixture of the purified Cdk5 protein and purified p25 obtained above was used as the enzyme source at the time of kinase activity measurement. Histone H1 (Sigma) was biotinylated and used as the substrate. Biotinylated histone was prepared by allowing histone to react with 10 equivalents of biotin (Ez-link Sulfo-NHS-LC-Biotin, Pierce) as molar ratio in the presence of 50 mM sodium bicarbonate at room temperature for 6 hours. Free biotin was removed by carrying out dialysis using TBS, and the resulting solution was used as the stock solution. Measurement of kinase activity was carried out in accordance with the SPA method (Amersham). The substrate, enzyme and γ ³³P-ATP were allowed to undergo the reaction at room temperature for 1 hour in a kinase reaction solution (50 mM HEPES-KOH, 10 mM MgCl₂, 2.5 mM EGTA, 0.1 mM PMSF, 1 mM Na₃VO₄, 1 mM NaF, 5 μg/ml aprotinin), and then the reaction was terminated by adding 3 volumes of a reaction termination solution (PBS containing 50 mM ATP, 5 mM EDTA, 1% Triton X-100 and 15 mg/ml streptavidin SPA beads) and stirring the mixture. This was allowed to stand at room temperature for 15 minutes and centrifuged (1,700 rpm, 2 min), and then amount of the isotope incorporated in the substrate was measured by Topcount (Beckman).

(3) Measurement of Kinase Inhibitory Activity of Alsterpaullone, Roscovitine and Compounds A, B, C and D:

Though chemical structures of Alsterpaullone and Roscovitine are completely different from each other, it is known that both are inhibitors selective for Cdk (Cancer Res. (1999) 59, 2566-2569, J. Med. Chem. (1999) 42, 2909-2919, Eur. J. Biochem. (1997) 243, 518-526, Eur. J. Biochem. (1997) 243, 527-536, and WO 99/65910). It is known that the compounds A, B, C and D are Cdk inhibitors (Bioorganic & Medicinal Chemistry Letters (1999) 9, 91-96, WO 97/16452, WO 99/43675). The inventors have verified the Cdk5 inhibitory activity of said compounds by the aforementioned method.

| | |
|---|---|
| Compound A | 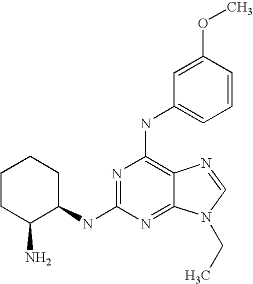 |
| Compound B | 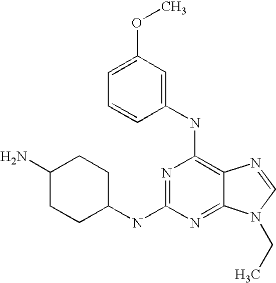 |
| Compound C | 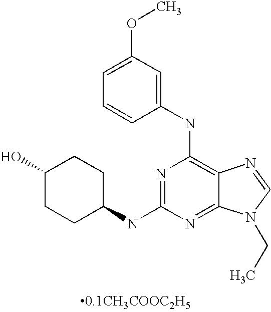 |
| Compound D | 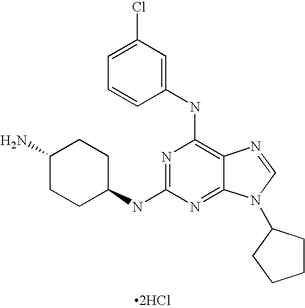 |

### EXAMPLE 3

Influence of Alsterpaullone, Roscovitine and compounds A, B, C and D on the Amount of APP Phosphorylation:

Hippocampus primary culture nerve cells on the 10th day of culturing were infected with APP₆₉₅ adenovirus, and 3 days thereafter, the medium was exchanged with a medium to which an appropriate concentration of Alsterpaullone (DMSO solution) or Roscovitine (DMSO solution) had been added. After additional 1 day of culturing, the cells were recovered using a cell lysis solution, and the degree of APP(Thr668) phosphorylation was determined by the method shown in Example 1. For example, the APP phosphorylation suppressing action of Alsterpaullone and Roscovitine was found at 10 μM and 50 μM, respectively. In this case, no changes were found in the expressed amount of total APP when detected with the anti-APP antibody 22C11. The APP phosphorylation suppressing action was also found in the compounds A, B, C and D in the same manner. In addition, said action was found also in a compound E or F having completely different structure from those of the aforementioned compounds.

### EXAMPLE 4

Influence of Alsterpaullone on the Aβ Production:

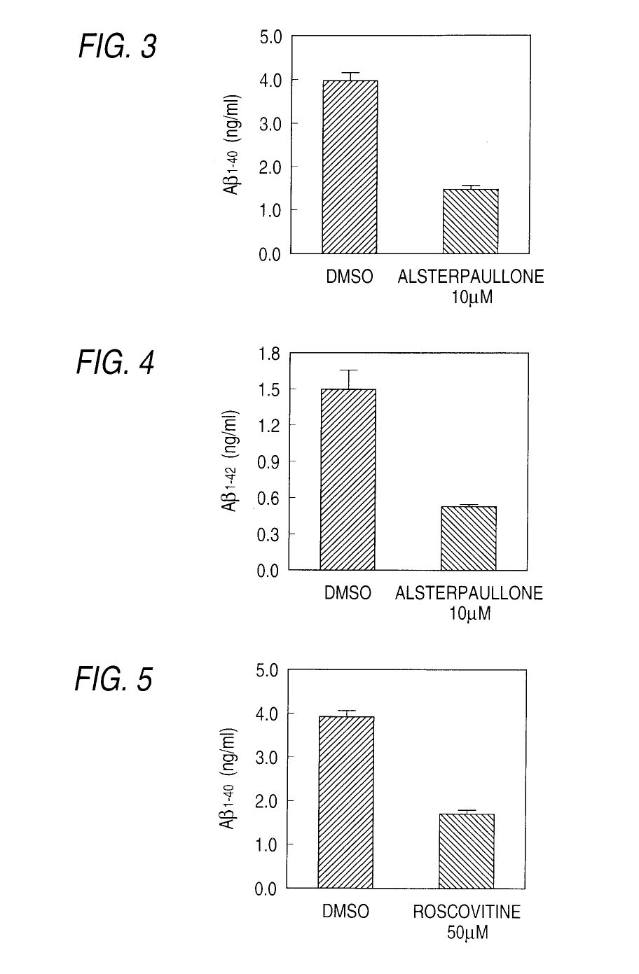

Hippocampus primary culture nerve cells on the 10th day of culturing were infected with APP₆₉₅ adenovirus, and 3 days thereafter, the medium was exchanged with a medium to which an appropriate concentration of Alsterpaullone (DMSO solution) had been added. After additional 1 day of culturing, the culture supernatant was recovered, and the amount of Aβ in the supernatant was determined by the method shown in Example 1. Alsterpaullone suppressed Aβ₁₋₄₀ production (FIG. 3) and Aβ₁₋₄₂ production (FIG. 4). As a control, DMSO was used instead of Alsterpaullone.

### EXAMPLE 5

Influence of Roscovitine and Compounds A, B, C and D on Aβ₁₋₄₀ Production:

In the same manner as in Example 4, amount of Aβ₁₋₄₀ in the supernatant was determined. The Aβ production suppressing effect of Roscovitine was confirmed (FIG. 5). The Aβ production suppressing effect of the compounds A, B, C and D was also confirmed. In addition, said effect was also found in the aforementioned compounds E and F.

Thus, from the tests using substances having the Cdk inhibitory action but completely different chemical structures, it was found that said substances can reduce the Aβ production. In addition, based on these test results and the fact that the Cdk isozyme which is known to have a physiological function in nerve cells is Cdk5 (J. Neurosci. (1999) 19, 6017-6026, and Genes & Dev. (1996) 10, 816-825), it was confirmed that said compounds exerted the Aβ production suppressing action by inhibiting the activity of Cdk5.

### EXAMPLE 6

Influence of Cdk5 Dominant Negative Mutant Adenovirus on Aβ Production

(1) Preparation of Cdk5 Dominant Negative Mutant Adenovirus Vector

A dominant negative mutant of Cdk5 (prepared by converting the 144th position aspartic acid residue into asparagine residue: Cdk5 D144N hereinafter (SEQ ID NO;15)) was prepared based on the description of Nikolic et al. (Genes & Development, 10, 816-825 (1996)). Quick Change Kit (Stratagene) was used in the introduction of mutation, and the actual operation was carried out in accordance with the instructions. As preparation for introducing a mutation, the Cdk5 gene described in Example 2 was used as the template, again amplified by PCR using a primer set of 5′-CTGAAGCTTCGCAGAAATACGAGAAACTGG-3′ (SEQ ID NO;16) and 5′-GATCTCGAGTAGGGCGGACAGAAGTCGGAG-3′ (SEQ ID NO;17) and introduced into pGEM-T Easy vector (Promega). The Cdk5 gene introduced into pGEM-T Easy vector was used as the template and amplified by PCR using a primer set of 5′-GGAGCTGAAATTGGCTAATTTTGGCCTGGCTCG-3′ (SEQ ID NO;18) and 5′-CGAGCCAGGCCAAAATTAGCCAATTTCAGCTCC-3′ (SEQ ID NO;19), and the product was treated with DpnI at 37° C. for 3 hours and then introduced into an E. coli strain JM-109 to obtain a mutation-introduced gene. After confirmation of its nucleotide sequence, in order to re-add the initiation methionine, the mutation gene was used as the template and amplified by PCR using a primer set of 5′-CTGAAGCTTATGCAGAAATACGAGAAACTGG-3′ (SEQ ID NO;20) and 5′-GATGTCGACTAGGGCGGACAGAAGTCGGAG-3′ (SEQ ID NO;21), and then cut out using the HindIII and SalI sites designed in the primers and introduced into a shuttle vector of ADEasy system, pShuttle. The pShuttle was used by introducing in advance a region of from CAG promoter to poly(A) addition signal (from PstI site to XhoI site) of the pCAG-pA vector described in Example 1. In order to facilitate insertion of the gene in carrying out the introduction, a multi-cloning site (a region which is cut out with BssHII) derived from pBluescript II-KS (Stratagene) was introduced into the HincII site existing between the CAG promoter and poly(A) addition signal. The shuttle vector introduced with the dominant negative mutant Cdk5 was made into linear strand using PmeI and introduced into an E. coli strain BJ5183 together with pADEasy-2 vector to prepare a virus genome recombinant plasmid. Preparation of adenovirus was carried out in the same manner as the case of APP adenovirus shown in Example 1.

(2) Influence of Cdk5 D144N Adenovirus on Aβ Production

Hippocampus primary culture nerve cells on the 7th day of culturing were infected with wild type APP₆₉₅ adenovirus together with the Cdk5 D144N adenovirus, the culture supernatant 2 days thereafter was recovered, and the amount of Aβ in the supernatant was determined. In comparison with a control, Cdk5 D144N suppressed the Aβ₁₋₄₀ production. As the control, cells infected with GFP-integrated adenovirus were used and compared in the same manner as in Example 1. Thus since similar Aβ production inhibitory activity found in the Cdk inhibitors was observed in the dominant negative mutant of Cdk5 capable of specifically suppressing the function of Cdk5, it was confirmed that the Aβ production can be suppressed by inhibiting the activity of Cdk5.

### INDUSTRIAL APPLICABILITY

Suppression of Aβ production can be achieved by the pharmaceutical composition of the invention. A disease based on said production suppression (e.g., AD) is characterized by pathological changes such as falling off of nerve cells, reduction of synapse numbers, senile plaque in the brain, accumulation of neurofibrillary change and the like, and senile plaque in the AD brain among them, namely accumulation of Aβ as the main composing molecule of the amyloid protein of senile plaque, is deeply concerned in the onset mechanism of AD, so that the invention is useful for anti-dementia drugs, anti-Alzheimer's disease drugs and the like.

Also, by detecting Aβ production by allowing a substance having Cdk, preferably Cdk5, inhibitory activity or a substance having APP threonine binding phosphoric acid phosphorylation inhibitory activity to contact with cells making use of the invention, for example, a substance having Aβ production suppressing action can be screened, and it can be used in diagnosis kits for dementia, Alzheimer's disease and the like.

1-10. (canceled)

11. A method for identifying a substance having β-amyloid production suppressing action, comprising:
(a) contacting a substrate with cyclin-dependent kinase (Cdk5) and Cdk5 inhibitor in the presence of ATP, and
(b) terminating the enzyme reaction and measuring the amount of phosphorylation of the substrate.

12. A method for identifying a substance having β-amyloid production suppressing action, comprising:
(a) contacting a substrate with cyclin-dependent kinase (Cdk5) and Cdk5 inhibitor in the presence of ATP,
(b) terminating the enzyme reaction and measuring the amount of phosphorylation of the substrate,
(c) contacting a cell with the Cdk5 inhibitor, and
(d) determining whether said Cdk5 inhibitor inhibits APP phosphorylation in the cell thereby identifying a compound having β-amyloid production suppressing action.

13. The method of claim 11 or 12, wherein said substrate is APP, histone H1 or their biotinylated forms.

14. A method of claim 12, wherein said ATP is isotope-labeled ATP.

15. The method of claim 12, wherein said inhibition of APP phosphorylation is determined by western blot using an antibody specific for phosphorylated APP.

16. The method of claim 12, wherein said cell does not proliferate.

17. The method claim 16, wherein said cell is a nerve cell.

18. A method for identifying a substance having β-amyloid production suppressing action, comprising:
(a) contacting a substrate with cyclin-dependent kinase (Cdk5) and a test compound in the presence of ATP, and
(b) terminating the enzyme reaction and measuring the amount of phosphorylation of the substrate.

19. A method for identifying a substance having β-amyloid production suppressing action, comprising:
(a) contacting a substrate with cyclin-dependent kinase (Cdk5) and a test compound in the presence of ATP,
(b) terminating the enzyme reaction and measuring the amount of phosphorylation of the substrate,
(c) contacting a cell with the test compound, and
(d) determining whether said test compound inhibits APP phosphorylation in the cell thereby identifying a compound having β-amyloid production suppressing action.

20. The method of claim 18 or 19, wherein said substrate is APP, histone H1 or their biotinylated form.

21. The method of claim 19, wherein said ATP is isotope-labeled ATP.

22. The method of claim 19, wherein said inhibition of APP phosphorylation is determined by western blot using an antibody specific for phosphorylated APP.

23. The method of claim 19, wherein said cell does not proliferate.

24. The method of claim 23, wherein said cell is a nerve cell.
